(19) United States
(12) Patent Application Publication
(10) Pub. No.: US 2011/0110858 A1
(43) Pub. Date: May 12, 2011

(54) Gold nanoparticle imaging agents and uses thereof

(76) Inventors: Omer Aras, Baltimore, MD (US); Thorsten Fleiter, Owings Mills, MD (US); Jean Jeudy, Baltimore, MD (US); Marie-Christine Daniel, Catonsville, MD (US)

(21) Appl. No.: 12/927,323

(22) Filed: Nov. 12, 2010

Related U.S. Application Data
(60) Provisional application No. 61/260,108, filed on Nov. 11, 2009.

(51) Int. Cl. A61K 49/00; C07D 207/16; A61K 49/04; A61K 49/06; B32B 5/16; B82Y 15/00

(52) U.S. Cl. 424/9.1; 548/533; 424/9.44; 424/9.3; 428/402; 977/773; 977/810; 977/927; 977/928; 977/930

(57) ABSTRACT

Overexpression of angiotensin-converting enzyme (ACE) has been associated with a number of pathophysiologies, including those associated with cancer and the cardiovascular system. Thus, targeted imaging of ACE is of crucial importance for monitoring tissue ACE activity as well as treatment efficacy. To this end, lisinopril-coated gold nanoparticles were prepared to provide a new type of probe for targeted molecular imaging of ACE by tuned K-edge computed tomography (CT) imaging.

### CROSS-REFERENCE TO RELATED APPLICATION

This nonprovisional application claims benefit of priority under 35 U.S.C. §119(e) of provisional applications U.S. Ser. No. 61/260,108, filed Nov. 11, 2009, now abandoned, the entirety of which are hereby incorporated by reference.

### BACKGROUND OF THE INVENTION

1. Field of the Invention

The present invention relates generally to the fields of nanoparticles and nanoparticle-based molecular imaging. More specifically, the present invention relates to gold nanoparticle imaging agents and uses thereof.

2. Description of the Related Art

Nanoparticle systems are promising new paradigms in pharmacotherapy and are being used in gene therapy, drug delivery, imaging, and drug discovery techniques. A goal of nanodiagnostics is to identify disease at its earliest stage, particularly at the molecular level. Nanoparticle-based molecular imaging has set a unique platform for cellular tracking, targeted diagnostic studies, and image monitored therapy.

Standard clinical imaging modalities such as CT, MRI, and ultrasound can be categorized as structural imaging modalities. These imaging modalities are able to identify anatomical patterns and to provide basic information regarding specific early disease process such as tumor location, size, and migration, based on endogenous contrast. However, these imaging modalities are not efficient in detecting tumors and metastases that are smaller than 0.5 cm, and they can barely distinguish between benign and cancerous tumors.

CT is not a molecular imaging modality since relevant targeted and molecularly specific imaging agents have not been developed. Present CT imaging agents are predominantly based on iodine containing molecules, which are effective in absorbing X-rays but are nonspecifically targeted because they cannot be conjugated to or otherwise associated with most biological components or cancer markers, and they allow only very short imaging times due to rapid clearance by the kidneys.

Therefore, the prior art is deficient in specifically targeted molecular imaging agents. The present invention fulfills this long-standing need and desire in the art.

### SUMMARY OF THE INVENTION

The present invention is directed to a drug labeled gold nanoparticle, wherein the drug interferes with the activity of the renin-angiotensin system. The present invention is further directed to a method of imaging an individual, comprising the steps of administering a plurality of the drug labeled gold nanoparticles described herein to the individual; and imaging the individual with a diagnostic device. In addition, the present invention is directed to a conjugate of an angiotensin converting enzyme inhibitor and a gold nanoparticle.

In one embodiment of the invention, there is provided an imaging agent of the invention is a renin-angiotensin system (RAS) targeted molecule. In specific embodiments, the gold nanoparticle is coated with the RAS targeted molecule). In further specific embodiments, a RAS targeted molecule is a metal-coated angiotensin converting enzyme (ACE) inhibitor. In other certain embodiments, a RAS targeted molecule of the invention is useful for generating imaging agents for diagnosing or monitoring disease. In other certain embodiments of the invention, a kit comprising a RAS targeted molecule of the invention useful for generating imaging agents for diagnosing or monitoring disease is provided.

### BRIEF DESCRIPTION OF DRAWINGS

FIG. 1 shows UV-vis spectra of citrate-coated gold nanoparticles and lisinopril-coated gold nanoparticles after dialysis (different dilutions were applied for each type of gold nanoparticle).

FIGS. 2A-2B show images and data of lisinopril-coated nanoparticles.

FIG. 2A: TEM image of lisinopril-coated nanoparticles. FIG. 2B: Size distribution of lisinopril-coated nanoparticles.

FIG. 3: DLS data showing the size distribution by volume of citrate-coated gold nanoparticles (first peak from left) and lisinopril-coated gold nanoparticles.

FIGS. 4A-4B show Zeta potential distribution data. FIG. 4A: Zeta potential distributions of citrate-coated gold nanoparticles. FIG. 4B: Zeta potential distributions of lisinopril-coated gold nanoparticles.

FIG. 5: DLS size distribution by intensity of lisinopril-capped gold nanoparticles i) alone with a dilution of 100, ii) with Tween 20 and in 1×PBS and iii) in 1×PBS.

FIG. 6: UV-vis absorption evolution in time of lisinopril-capped gold nanoparticles a) with a dilution of 150 in 1×PBS, b) with a dilution of 100, with Tween 20 and in 1×PBS.

FIG. 7: In vivo CT images of a rat after tail vein injection of 100 μL of 0.68 μM lisinopril-capped GNPs. Left: gray scale image and right: 3D-volume rendered image.

FIG. 8 shows the derivatization of enalapril.

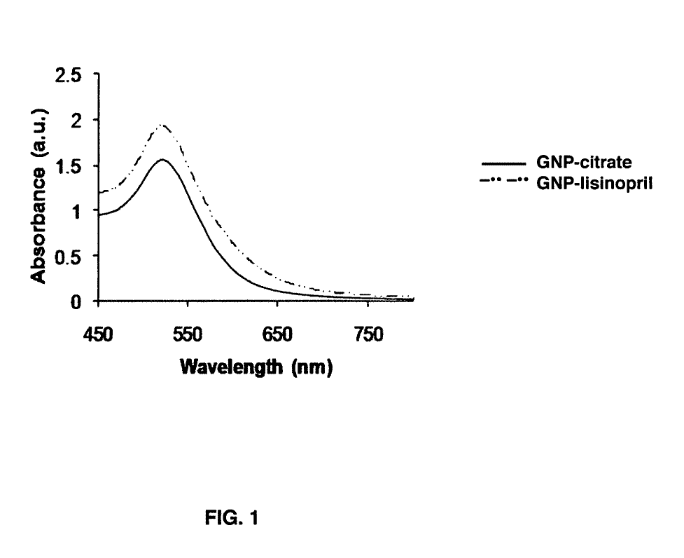

### DETAILED DESCRIPTION OF THE INVENTION

As described in detail below, the present invention is directed to a drug labeled gold nanoparticle, wherein said drug interferes with the activity of the renin-angiotensin system. Representative drugs which interfere with the activity of the renin-angiotensin system include but are not limited to an angiotensin converting enzyme inhibitor and an angiotensin II receptor antagonist. Angiotensin converting enzyme inhibitors are well known in the art and representative examples include but are not limited to lisinopril, enalapril, captopril, fosinopril, quinapril, ramipril, trandolapril, benazepril, moexipril and perindopril. Angiotensin II receptor antagonists are also well known in the art and representative examples include but are not limited to candesartan, eprosartan, irbesartan, losartan, olmesartan, telmisartan, and valsartan. Depending upon the type of drug used to label the gold nanoparticle, the drug may need to be attached to the gold nanoparticle via a capping agent. Representative capping agents include but are not limited to agents which contain a phosphine group, an amine group or a thiol group. As is well known to those having ordinary skill in this art, nanoparticles typically have a diameter of from about 1 nm to about 100 nm. Preferably, the drug labeled gold nanoparticle of the present invention have a diameter of from about 10 nm to about 50 nm and most preferably about 30 nm. As designed, the drug labeled gold nanoparticles of the present invention have a circulation time of from about 2 hours to about 6 hours and preferably the circulation time is about 4 hours.

A stealth agent can also be incorporated to the drug labeled gold nanoparticles, in order to improve the circulation time and the biocompatibility of the invention. Representative stealth agents include but are not limited to oligo(ethylene glycol) derivatives and polyethylene glycol) derivatives. It is further contemplated that the drug coated gold nanparticles may further contain a fluorescence imaging agent to permit multifunctional use (fluorescent and paramagnetic), e.g., combination of CT and MRI.

In another embodiment of the present invention, there is provided a method of imaging an individual, comprising the steps of administering a plurality of the drug labeled gold nanoparticles described herein to said individual; and imaging the individual with a diagnostic device. In one aspect, this method may further comprise monitoring delivery of the drug labeled gold nanoparticles to the individual with the diagnostic device; and diagnosing or monitoring the status of the individual. Representative diagnostic devices employed by one in this art include but are not limited to an imaging method selected from the group consisting of MRI, optical imaging, optical coherence tomography, X ray, computed tomography, positron emission tomography, or combinations thereof. Using this method of the present invention, one may diagnose or monitor (including monitoring disease state, disease progress or efficacy of drug treatment) an individual that has or is at risk for heart failure, myocardial ischemia, hypertension, atherosclerosis, diabetic nephropathy, or cancer. Representative drugs useful in this method include but are not limited to angiotensin converting enzyme inhibitors and an angiotensin II receptor antagonists. Representative angiotensin converting enzyme inhibitor include lisinopril, enalapril, captopril, fosinopril, quinapril, ramipril, trandolapril, benazepril, moexipril and perindopril. Representative angiotensin II receptor antagonists include candesartan, eprosartan, irbesartan, losartan, olmesartan, telmisartan, and valsartan. As is described above, it may be beneficial or necessary for the drug to be attached to the gold nanoparticle via a capping agent. Representative capping agents include but are not limited to agents which contain a phosphine group, an amine group or a thiol group. The size and circulation times of the drug labeled gold nanoparticles is described above. If a cancer is monitored or imaged in the individual, it is contemplated that the cancer would have a diameter or area of from about 0.1 mm to about 10 mm. In one preferred embodiment, the computed tomography method is spectral computed tomography.

In another embodiment of the present invention, there is provided a kit, comprising the drug labeled gold nanoparticle of the present invention. As described below, the drug labeled gold nanoparticle may be contained in the kit in a pharmaceutically acceptable formulation that can be administered to a mammal.

In yet another embodiment of the present invention, there is provided a conjugate of an angiotensin converting enzyme inhibitor and a gold nanoparticle. In preferred forms of this conjugate, the angiotensin converting enzyme inhibitor is selected from the group consisting of lisinopril, enalapril, captopril, fosinopril, quinapril, ramipril, trandolapril, benazepril, moexipril, and perindopril. In certain aspects of this conjugate, the angiotensin converting enzyme inhibitor is attached to said gold nanoparticle via a capping agent. In a preferred form, the capping agent contains a phosphine group, an amine group or a thiol group. Generally, the nanoparticle has a diameter of from about 10 nm to about 50 nm and has a circulation time of from about 2 hours to about 6 hours.

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, a “RAS targeted molecule” (or an apparent derivation thereof) is a molecule that binds to a component of the RAS system, including for example, an agonist, partial agonist, antagonist, or other binding partner of ACE or an angiotensiogen receptors (including, for example, AT1 or AT2). RAS targeted molecule can be a molecule comprising more than one molecule (e.g., lisinopril-coated gold nanoparticles or enalapril-coated gold nanoparticles).

In certain embodiments of the invention, the inventors have discovered that certain RAS targeted molecules can be useful for generating imaging agents for diagnosing or monitoring cardiovascular and renal pathophysiologies (including, for example, heart failure, myocardial ischemia, hypertension, and diabetic nephropathy), and cancer.

In other particular aspects of the invention drawn to RAS targeted molecules useful for generating imaging agents, the RAS targeted molecule targets an ANG II receptor. In other particular aspects an ANG II antagonist includes, for example, candesartan, eprosartan, irbesartan, losartan, olmesartan, telmisartan, and valsartan.

In particular aspects of the invention drawn to diagnosing or monitoring cancer, the cancer is a cancer not detectable by convention imaging modalities. In certain aspects, the cancer is too small to be detected by convention imaging modalities. In further aspects the cancer has a diameter or area of about 10 mm to about 0.1 mm. In other further aspects, the cancer has a diameter or area of about 10 mm to about 0.2, about 10 mm to about 0.3 mm, about 10 mm to about 0.4 mm, about 10 mm to about 0.5 mm, about 10 mm to about 0.6 mm, about 10 mm to about 0.7 mm, about 10 mm to about 0.8 mm, about 10 mm to about 0.9 mm, about 10 mm to about 1.0 mm, about 10 mm to about 1.1, about 10 mm to about 1.2, about 10 mm to about 1.3 mm, about 10 mm to about 1.4 mm, about 10 mm to about 1.5 mm, about 10 mm to about 1.6 mm, about 10 mm to about 1.7 mm, about 10 mm to about 1.8 mm, about 10 mm to about 1.9 mm, about 10 mm to about 2.0 mm, about 10 mm to about 2.1, about 10 mm to about 2.2, about 10 mm to about 2.3 mm, about 10 mm to about 2.4 mm, about 10 mm to about 2.5 mm, about 10 mm to about 2.6 mm, about 10 mm to about 2.7 mm, about 10 mm to about 2.8 mm, about 10 mm to about 2.9 mm, about 10 mm to about 3.0 mm, about 10 mm to about 3.1, about 10 mm to about 3.2, about 10 mm to about 3.3 mm, about 10 mm to about 3.4 mm, about 10 mm to about 3.5 mm, about 10 mm to about 3.6 mm, about 10 mm to about 3.7 mm, about 10 mm to about 3.8 mm, about 10 mm to about 3.9 mm, about 10 mm to about 4.0 mm, about 10 mm to about 4.1, about 10 mm to about 4.2, about 10 mm to about 4.3 mm, about 10 mm to about 4.4 mm, about 10 mm to about 4.5 mm, about 10 mm to about 4.6 mm, about 10 mm to about 4.7 mm, about 10 mm to about 4.8 mm, about 10 mm to about 4.9 mm, an about 10 mm to about 5.0 mm. In other further aspects, the cancer has a diameter or area of about 10 mm, about 9 mm, about 8 mm, about 7 mm, about 6 mm, about 5 mm, about 4 mm, about 3 mm, about 2 mm, about 1 mm. In other further aspects, the cancer has a diameter or area less than 5 mm.

In other particular aspects of the invention drawn to diagnosing or monitoring cancer, cancer refers to, a pathophysiological condition whereby a cell or cells is characterized by dysregulated and/or proliferative cellular growth into adjacent tissue or at distal sites through metastasis, in both, an adult or child, which includes, but is not limited to, carcinomas and sarcomas, such as, for example, acute lymphoblastic leukemia, acute myeloid leukemia, adrenocortical cancer, AIDS-related cancers, AIDS-related lymphoma, anal cancer, astrocytoma (including, for example, cerebellar and cerebral), basal cell carcinoma, bile duct cancer, bladder cancer, bone cancer, brain stem glioma, brain tumor (including, for example, ependymoma, medulloblastoma, supratentorial primitive neuroectodermal, visual pathway and hypothalamic glioma), cerebral astrocytoma/malignant glioma, breast cancer, bronchial adenomas/carcinoids, Burkitt's lymphoma, carcinoid tumor (including, for example, gastrointestinal), carcinoma of unknown primary site, central nervous system lymphoma, cervical cancer, chronic lymphocytic leukemia, chronic myelogenous leukemia, chronic myeloproliferative disorders, colon cancer, colorectal cancer, cutaneous T-Cell lymphoma, endometrial cancer, ependymoma, esophageal cancer, Ewing's Family of tumors, extrahepatic bile duct cancer, eye cancer (including, for example, intraocular melanoma, retinoblastoma, gallbladder cancer, gastric cancer, gastrointestinal carcinoid tumor, gastrointestinal stromal tumor (GIST), germ cell tumor (including, for example, extracranial, extragonadal, ovarian), gestational trophoblastic tumor, glioma, hairy cell leukemia, head and neck cancer, squamous cell head and neck cancer, hepatocellular cancer, Hodgkin's lymphoma, hypopharyngeal cancer, islet cell carcinoma (including, for example, endocrine pancreas), Kaposi's sarcoma, laryngeal cancer, leukemia, lip and oral cavity cancer, liver cancer, lung cancer (including, for example, non-small cell), lymphoma, macroglobulinemia, malignant fibrous histiocytoma of bone/osteosarcoma, medulloblastoma, melanoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, myeloma, nasal cavity and paranasal sinus cancer, nasopharyngeal cancer, neuroblastoma, non-Hodgkin's lymphoma, oral cancer, oral cavity cancer, osteosarcoma, oropharyngeal cancer, ovarian cancer (including, for example, ovarian epithelial cancer, germ cell tumor), ovarian low malignant potential tumor, pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, prostate cancer, rectal cancer, retinoblastoma, rhabdomyosarcoma, salivary gland cancer, soft tissue sarcoma, uterine sarcoma, Sézary syndrome, skin cancer (including, for example, non-melanoma or melanoma), small intestine cancer, supratentorial primitive neuroectodermal tumors, T-Cell lymphoma, testicular cancer, throat cancer, thymoma, thymoma and thymic carcinoma, thyroid cancer, transitional cell cancer of the renal pelvis and ureter, trophoblastic tumor (including, for example, gestational), unusual cancers of childhood and adulthood, urethral cancer, endometrial uterine cancer, uterine sarcoma, vaginal cancer, viral induced cancers (including, for example, HPV induced cancer), vulvar cancer, Waldenström's macroglobulinemia, Wilms' Tumor, and women's cancers.

### Kits of the Invention

Any of the molecules described herein (e.g., a gold-labeled ACE inhibitor) may be contained in a kit. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional component may be separately placed. However, various combinations of components may be comprised in a vial. The kits of the present invention also will typically include a means for containing the molecule and any other reagent containers in close confinement for commercial sale. Such containers may include injection or blow molded plastic containers into which the desired vials are retained. Kits of the present invention include kits comprising a gold-labeled ACE useful as an imaging agent. Such kits will generally contain, in suitable container means, a pharmaceutically acceptable formulation that can be administered to a mammal. The kit may have a single container means and/or it may have distinct container means.

When the components of the kit are provided in one and/or more liquid solutions, the liquid solution is an aqueous solution, with a sterile aqueous solution being particularly preferred. The gold-labeled ACE useful as an imaging agent may also be formulated into a syringeable composition. In which case, the container means itself may be a syringe, pipette, and/or other such like apparatus, from which the formulation may injected or otherwise administered to a mammal, and/or even applied to and/or mixed with the other components of the kit. Examples of aqueous solutions include, but are not limited to ethanol, DMSO and/or Ringer's solution. In certain embodiments, the concentration of DMSO or ethanol that is used is no greater than 0.1% or (1 ml/1000 L).

The components of the kit may also be provided as a dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the gold-labeled ACE useful as an imaging agent is suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the agents or compounds within the body of a mammal. Such an instrument may be a syringe, pipette, forceps, and/or any such medically approved delivery vehicle.

### Example 1

### Materials

All glassware used was cleaned with freshly prepared aqua regia solution (HCl/HNO₃, 3:1), then rinsed thoroughly with ultrapure water before use. The water was distilled and subsequently purified to 18 mΩ ultrapure water quality using the Milli-Q academic system. Gold chloride (HAuCl₄.H₂O) and trisodium citrate (Na₃C₆H₅O₇.2H₂O), obtained from Electron Microscopy Sciences, and sodium hydroxide (NaOH), from Fisher Scientific, were of analytical grade and were used without further purification. Tween 20 was purchased from Aldrich. Lisinopril dehydrate was obtained from Waterstone Technology LCC. The Millex-LCR 0.45 μm syringe filters were obtained from Millipore. The TEM carbon coated 200 mesh copper grids were purchased from Ted Pella, Inc.

### Example 2

### Preparation of Gold Nanoparticles

The synthesis was performed using a modified Frens method^{[23]}. In a 1 L 2-neck-round-bottom flask equipped with a condenser, 500 mL of 0.5 mM HAuCl₄ in ultrapure water was brought to boil. Rapid addition of 26 mL of 5% sodium citrate solution (n_{citrate}/n_{Au}=17.9) to the vigorously stirred gold chloride solution resulted in a series of color change from pale yellow to colorless, then light purple, deeper purple, turning reddish and finally to dark wine red. The color change occurred over 7 minutes. The solution was maintained for 15 minutes at boiling temperature and then removed from the heating bath. Stirring was continued until the solution cooled down to room temperature. The size of the prepared gold nanoparticles was 15.8 nm±1.9 nm according to the TEM measurements.

### Example 3

### Preparation of Concentrated Solution of Lisinopril-Coated Gold Nanoparticles

Lisinopril dehydrate (330 mg, molar ratio of lisinopril/GNP: 4.02×10⁵) was dissolved in 10 mL of water adjusted to pH 11 using 2 N sodium hydroxide.^{[24]} The resulted lisinopril solution was added to a solution (525 mL) of citrate-stabilized GNP solution previously prepared (15.8 nm, 3.54 nM) while strongly stirring in a 1 L beaker. A certain amount of 2 N NaOH was then added to keep pH around 11. The reaction was kept under strong stirring overnight at room temperature. The lisinopril-capped gold nanoparticles solution was centrifuged several times at 31,000 g for 45 min. at 4° C. and redispersed in basic water (pH 8-9) in order to remove the excess of lisinopril and the exchanged citrate molecules. This purified lisinopril-capped gold nanoparticles solution was used for all the characterization techniques. Concentration of these lisinopril-capped gold nanoparticles was performed by centrifugation at 16000 rpm for 30 min. at 4° C. and recovery of the pellet (2.5 mL).

Gold nanoparticles of 30 nm diameter were prepared. The size of 30 nm was chosen for providing a much longer circulation time (>4 h) in CT imaging with respect to iodine (<10 minutes). The formation of gold nanoparticles are confirmed by UV-vis spectroscopy. The excitation of this surface plasmon by light produces a strong absorption in visible range (around 500 nm), showing a surface plasmon resonance (SPR) peak characteristic of gold nanoparticle. The exact position of the SPR peak can also be used to evaluate the size of the gold nanoparticles, and it is sensitive to coating change. The mean overall size of the gold nanoparticles (core+ligand) can also be assessed by dynamic light scattering (DLS).

### Example 4

### Characterization

UV-vis absorption spectra were recorded on a DU 730 UV/Vis life science spectrophotometer. Dynamic light scattering (DLS) data were obtained by using a Zetasizer Nano-ZS particle characterization system. Transmission electron micrographs (TEM) were taken by using a Tecnai T12 electron microscope equipped with an ATM digital camera. The pictures acquired were analyzed using SigmaScan Pro software to obtain the mean size of the gold nanoparticles and the associated standard deviation. The Fourier-transform infrared (FTIR) spectra were recorded using Paragon 500 FT-IR (Perkin Elmer) instrument equipped with a ZnSe attenuated total reflectance (ATR) crystal. A drop of the dissolved lisinopril or lisinopril-coated gold nanoparticle solution was casted on the ATR crystal and the FTIR spectra were recorded after evaporation of the solvent to create a film on the crystal surface.

### Example 5

### In vivo CT Imaging

As CT contrast agent, 100 μL of concentrated lisinopril-capped GNPs were injected into the tail of a rat and imaging was performed shortly after injection. CT data were acquired using a Philips Brilliance CT 64-slice (Philips Healthcare, Andover, Mass.). Imaging parameters were as follows: slice thickness, 0.625 mm; pitch, 0.984:1; 80 kVp, 350 mA; field of view, 512×512, gantry rotation time, 0.4 s; table speed, 40 mm/rotation. All animals were scanned in the cranial to caudal direction from the head to the tail. 3D images were also obtained by using TeraRecon-Aquarius software (San Mateo, Calif.).

### Example 6

### Lisinopril-Coated Gold Nanoparticles

The stabilization of gold nanoparticles originates from the Au/ligand interaction at the gold particles surface. However, the interaction of gold nanoparticles surface is increasingly stronger with, for example, phosphine, amine and thiol functions. Thus capping agents containing phosphine, amine or thiol groups will impart gold nanoparticles with respectively increased stability.

Since lisinopril contains a primary amine, it produces stable gold-labeled nanoparticles by direct interaction of its amine with the gold nanoparticles surface atoms. The ligand exchange reaction is performed at pH 11 to ensure an overall negative charge on lisinopril, since it was shown to be important for the new ligand to be negatively charged. The pKa values of lisinopril at 25° C. are 2.5 and 4.0 for the central CO2H and the prolyl CO2H respectively, 6.7 for the secondary amine group and 10.1 for the lysyl amino group.

A first indication of the replacement of citrate by lisinopril molecules on the gold nanoparticles surface consisted of a simple test: the effect of salt concentration. The stability was tested at a physiological salt concentration (150 mM NaCl) and even at higher concentrations to determine the critical coagulation concentration (ccc). Since amine-stabilized gold nanoparticles were found to present stability similar to the thiol-capped gold nanoparticles, the former are expected to be stable at physiological salt concentration or higher. Considering that an embodiment of the invention is in vivo imaging using the lisinopril-coated gold nanoparticles, their stability in a physiological salt concentration (150 mM NaCl) was demonstrated. After the addition of salt, the lisinopril-coated gold nanoparticles showed no sign of aggregation, even few hours after the salt addition, demonstrating the increased stability of the gold nanoparticles provided by this new ligand. This result confirms the exchange of citrate with lisinopril and opens the possibility of using the lisinopril-coated gold nanoparticles as in vivo imaging agent.

The SPR peak of the gold nanoparticles is sensitive to their size, but also their coating and polydispersity. A comparison of the two absorption spectra obtained for citrate-coated gold nanoparticles and lisinopril-coated gold nanoparticles respectively gives another indication of the gold nanoparticles modification with lisinopril. The assessment of the size and shape of the gold nanoparticles after modification was performed using TEM. The mean overall size of the gold nanoparticles modification with lisinopril was also assessed by DLS. FTIR spectra of pure lisinopril and lisinopril-coated gold nanoparticles respectively were compared. The gold nanoparticles concentration was evaluated from the absorbance at 450 nm in its absorption spectrum, and using the size measured on the TEM pictures.

The lisinopril concentration was obtained as follows. First, to avoid the interference of the gold nanoparticles core during the quantification of lisinopril, the latter was stripped from the gold nanoparticles by exchange with mercaptoethanol. An excess of mercaptoethanol was added to lisinopril-coated gold nanoparticles and stirred for 2 hours to ensure a complete ligand exchange. The resulted mercaptoethanol-coated gold nanoparticles are centrifuged. The supernatant containing lisinopril and the excess of mercaptoethanol are then analyzed using LC-MS, and the lisinopril concentration was obtained by comparison with calibration curves.

### Example 7

### Absorption Spectra of the Gold Nanoparticles

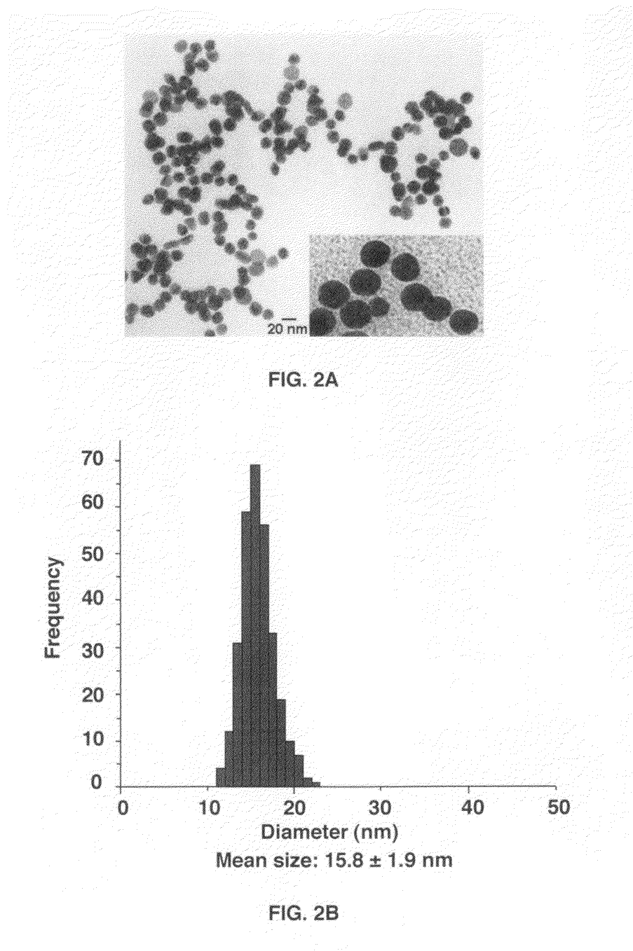

Gold nanoparticles exhibit surface plasmon that is sensitive to the size, coating and polydispersity of gold nanoparticles in solution. Hence, some information about gold nanoparticles can be obtained through the examination of the surface plasmon resonance (SPR) peak present on their absorption spectra. A comparison of the two absorption spectra obtained for citrate-coated gold nanoparticles and lisinopril-capped gold nanoparticles respectively gives another indication of the gold nanoparticles modification with lisinopril. Indeed, the SPR peaks for citrate-coated gold nanoparticles and lisinopril-capped gold nanoparticles respectively occurred at 519 nm and 526 nm. This small difference of 7 nm between the two SPR peaks is characteristic of the change of coating of the gold nanoparticles. Also, the size of citrate-coated gold nanoparticles can be estimated using the ratio Aλmax/Aλ450. In this way, the diameter of the citrate-coated gold nanoparticles was estimated to be 16 nm. This estimation technique applies only for gold nanoparticles coated with citrate, so it cannot be used for the estimation of the lisinopril-coated gold nanoparticles size. Since the SPR peak of the latter nanoparticles showed only a slight shift with respect to the one of the former nanoparticles, the size of gold nanoparticles is not expected to have changed during ligand exchange reaction. This was confirmed by analysis of TEM (see FIG. 2A).

The TEM pictures of lisinopril-coated gold nanoparticles showed well dispersed particles with a mean core size of 14.7 nm±2.1 nm (FIG. 2B). This value coincides with the estimated size of the citrate-coated gold nanoparticles obtained from the absorption spectrum. Thus, the size of the particles did not change during ligand exchange, as it could also be interpreted from the absorption spectrum of lisinopril-coate gold nanoparticles. Consequently, the large increase in total size observed in the DLS spectrum of lisinopril-coated gold nanoparticles is challenging to explain. The increase in size of 19 nm has to be attributed only to the lisinopril coating, which means that the layer thickness is around 9-10 nm. This value is much larger than the size of a molecule of lisinopril, so it cannot correspond to a monolayer of lisinopril, but rather multilayers.

### Example 8

### Gold Nanoparticles Size Measurements

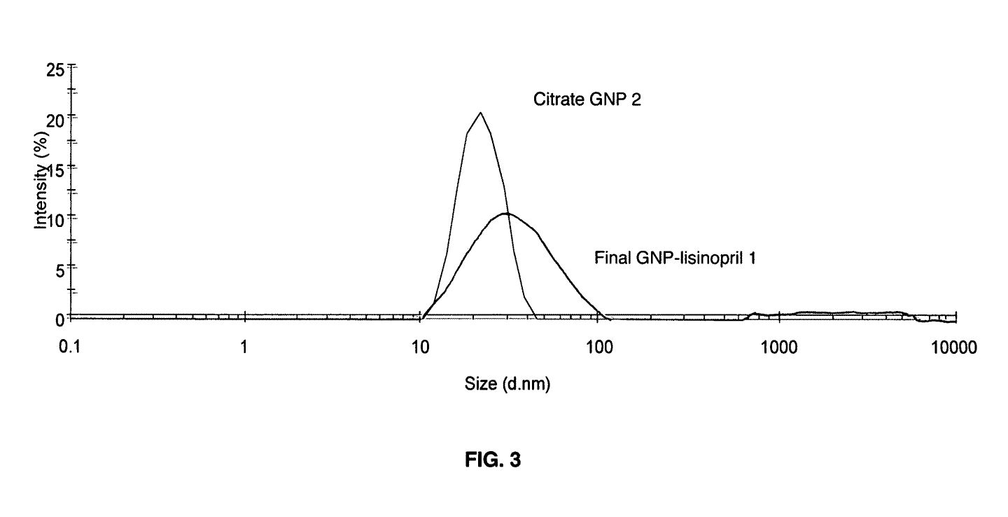

Images of the gold nanoparticles are taken using a transmission electron microscope. The sizes of gold nanoparticles (before and after citrate exchange) were measured using dynamic light scattering (DLS) and transmission electron microscopy (TEM). DLS measurements were performed for both gold nanoparticle types (FIG. 3). The hydrodynamic diameters obtained are 18.16 nm and 37.40 nm for citrate-coated gold nanoparticles and lisinopril-coated gold nanoparticles, respectively. These DLS values are including the ligand layers, so they can provide an estimation of the thickness of the capping layer if the size of the gold particle core is known. Concerning the citrate-coated gold nanoparticles, the diameter of 18 nm including citrates is consistent with the estimated size of 16 nm from the UV-vis data. Regarding the lisinopril-coated gold nanoparticles, the diameter of 37 nm shows an increase of 19 nm for the total size of the particles. This increase in size can result either from a growth of the gold nanoparticles core and the change of ligand, or only from the new ligand.

### Example 9

### Concentration and Charge Density of Lisinopril Capped-Gold Nanoparticles

Since both the TEM size measurements and the UV-vis spectrum indicated that the size of the gold nanoparticles remained the same before and after lisinopril modification, the extinction coefficient reported for 15 nm citrate-coated gold nanoparticles can be used to find the final concentration (after purification) of lisinopril-capped gold nanoparticles. Since the extinction coefficient c450 is 2.18×108 M-1 cm-1 for 15 nm citrate-coated gold nanoparticles, and the absorbance at 450 nm was 0.423 from its absorption spectrum, the concentration of the lisinopril-coated gold nanoparticles is 1.94 nM. This corresponds to a dilution phenomenon (initial concentration of citrate-coated gold nanoparticles: 2.8 nM) that might have occurred during the dialysis process.

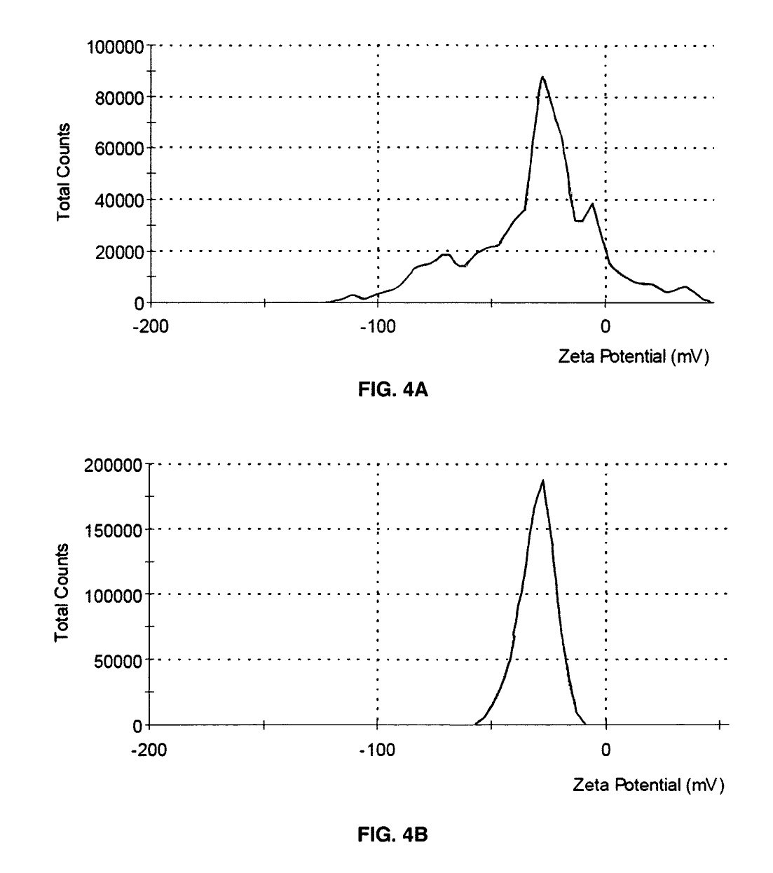

The overall surface charge on the gold nanoparticles was also assessed. The zeta potential was measured for the gold nanoparticles before and after modification. The zeta potential reports indicated a similar zeta potential value of around −30 mV for both citrate-coated gold nanoparticles and lisinopril-capped gold nanoparticles, which is expected for negatively charged gold nanoparticles of this size (FIG. 4). But the zeta deviations and the conductivities showed main differences between the two types of gold nanoparticles. Indeed, the zeta deviation was much higher for the citrate-coated gold nanoparticles (27 mV) than for the lisinopril-capped gold nanoparticles (7.7 mV). Also, the conductivity measured was two orders of magnitude higher for the citrate-coated gold nanoparticles (0.510 mS/cm) than for the lisinopril-coated gold nanoparticles (0.007 mS/cm).

### Example 10

### Stability in PBS Buffer

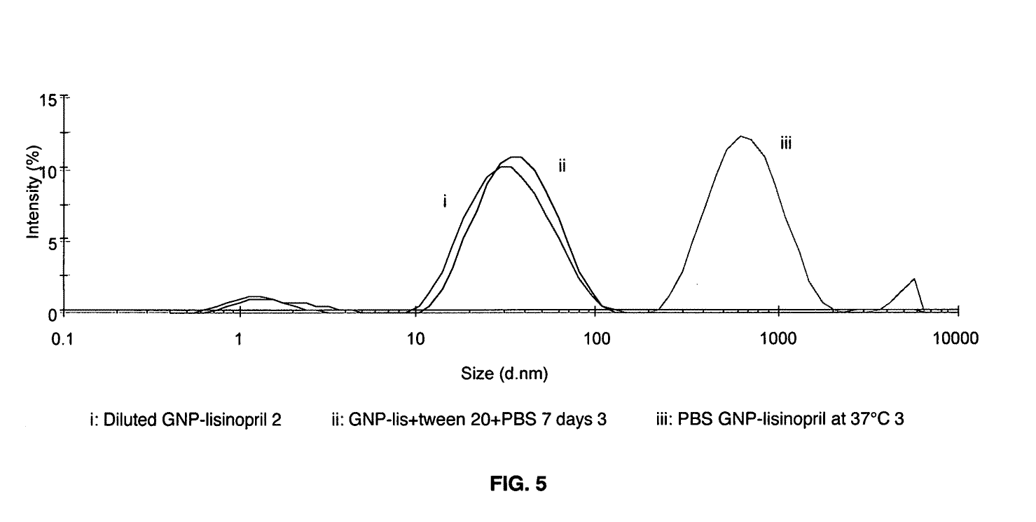

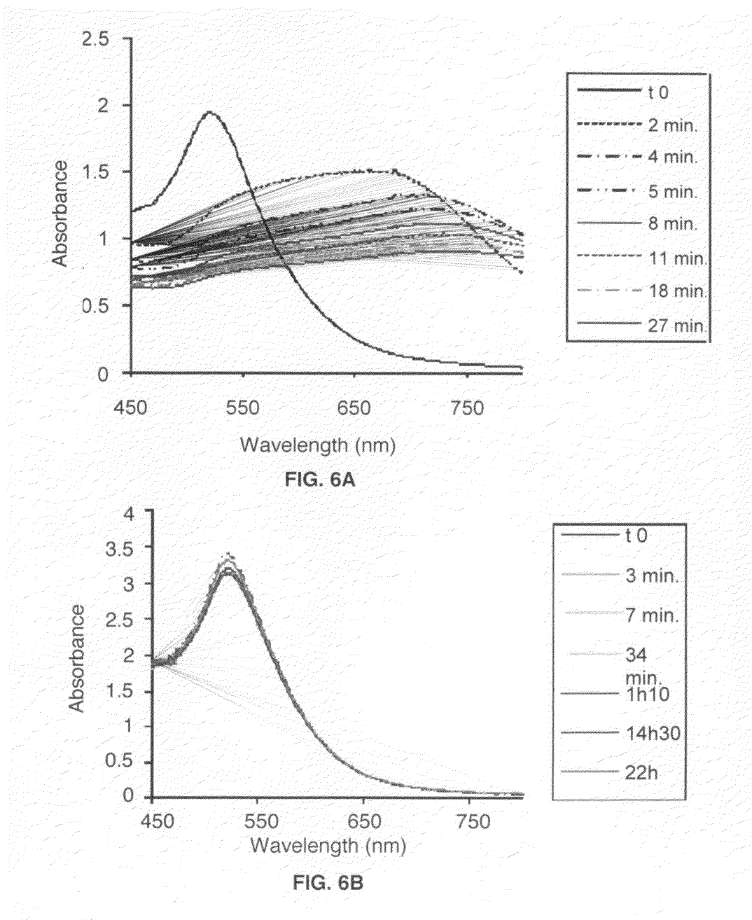

The evolution of the lisinopril-capped GNPs was followed in 1×PBS (Phosphate Buffer Saline) in order to investigate their stability in biological media. This study was performed by means of UV-vis absorption spectrum and DLS. A first trial involved 0.2 mL of 10×PBS added 1.8 mL of 150 times diluted GNPs. The addition of PBS buffer provoked immediately a visible change of color from red to blue-purple. The spectroscopic changes were recorded by UV-vis absorption and DLS (FIGS. 5 and 6A-6B). Very quickly, the surface plasmon resonance band shifted to around 700 nm and then to 750 nm (FIG. 6A); also the hydrodynamic diameter increased to around 700 nm. These phenomena both reflect large and irreversible aggregation. This indicates that the lisinopril-capped GNPs have limited stability in the presence of salt and some of the lisinopril molecules are displaced from the gold surface, meaning that the amine/gold interaction is not strong enough to endow the particles with the highest stability in buffers.

In order to improve the stability of the lisinopril-capped gold nanoparticles in salty environment, some Tween20 (20 mg) was added to the gold nanoparticles solution. The appropriate volume of 10×PBS was then added to reach a concentration of 1×PBS, and the evolution was followed again by UV-vis absorption and by DLS. As observed, the gold nanoparticles remained stable for at least seven days in 1×PBS when Tween 20 is present: the surface plasmon resonance band and the hydrodynamic diameter are very minimally affected. This result exhibits the role of Tween 20 as stabilizer of suspensions and promotes its use for stability enhancement of these gold nanoparticles.

### Example 11

### In Vivo CT Imaging of Lisinopril-Capped GNPs

The lisinopril-capped gold nanoparticles have been prepared for use as CT tracers of tissue ACE. In order to investigate their ability for such a role, a first in vivo experiment was performed on a rat model. A tail vein injection was carried out using 100 μL of the concentrated lisinopril-capped gold nanoparticles (0.68 μM gold nanoparticles, or 20 mg Au/mL), which corresponds to 0.068 nM of gold nanoparticles or 2 mg of Au.

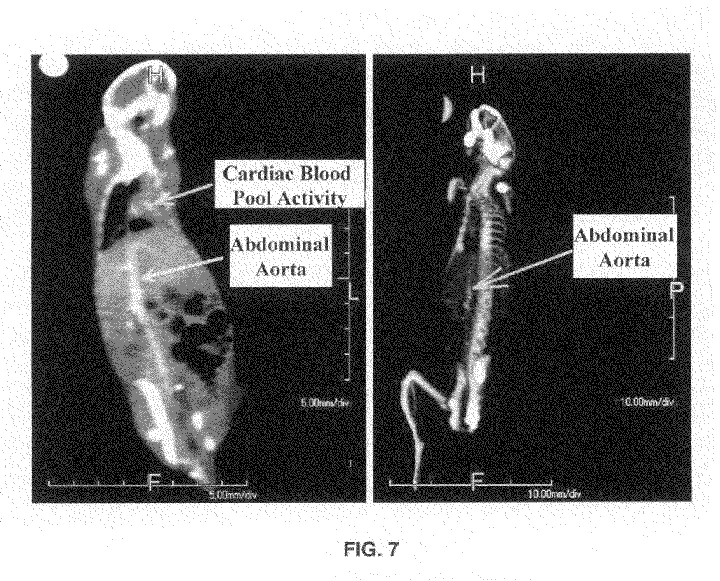

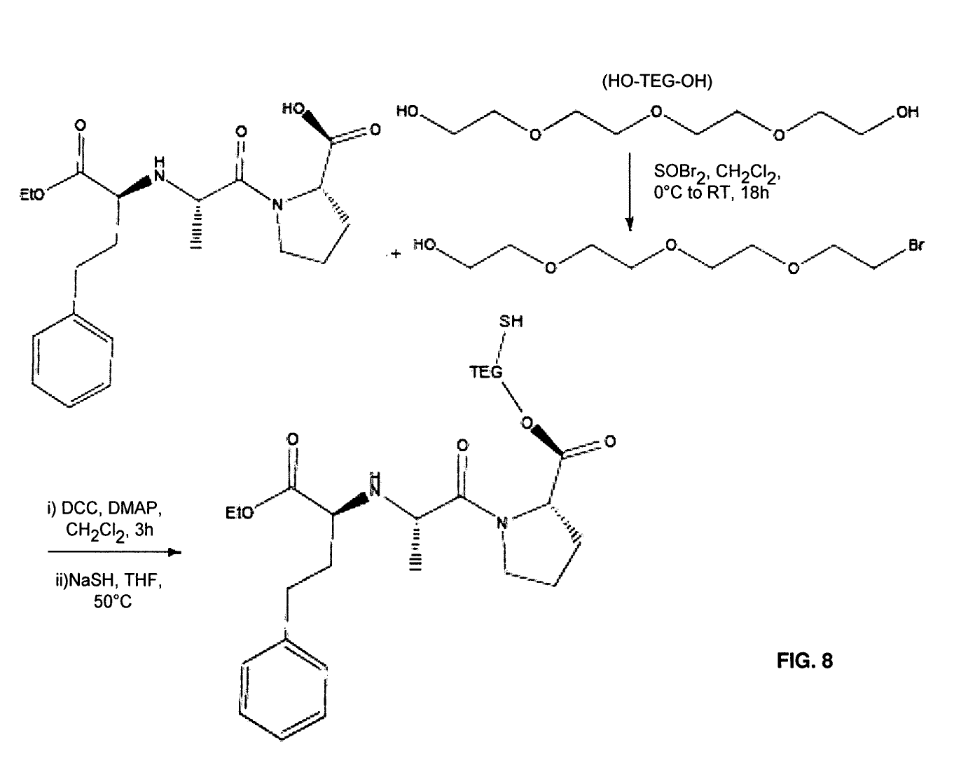

FIG. 7 shows the CT scans acquired during the first-pass of the contrast agent. At this stage, only the arterial phase is enhanced. It can be noticed that enough contrast is produced by the 0.68 μM gold nanoparticles to enable the visualization of the abdominal aorta as well as the cardiac blood pool activity. With time, the gold nanoparticles were observed to accumulate in the kidneys but also to remain around the lungs. Since the lungs are known to express tissue ACE, this slow localization around the lungs could be the consequence of binding of the lisinopril-capped gold nanoparticles with the ACE enzyme, thus limiting the renal excretion of these bound gold nanoparticles. These data support the use of lisinopril-capped gold nanoparticles as tracers for tissue ACE imaging.

### Example 12

### Enalapril-Coated Gold Nanoparticles

Enalapril is first derivatized in a derivatization performed in three steps. First, bromated tetraethylene glycol is obtained in one step from tetraethylene glycol and thionyl bromide (Hurley, et al., 2008, Organic & Biomolecular Chemistry, 6(14), pp. 2554-2559.). Then the bromated tetraethylene glycol is attached to enalapril through esterification of its carboxylic acid using dicyclocarbodiimide (DCC) and 4-N,N-dimethylaminopyridine (DMAP) as a catalyst (Neises, et al., 1978, Angewandte Chemie, 90(7), pp. 556-557.). Eventually the thiol group is obtained at the end of the tetraethylene glycol spacer by the use of sodium sulfhydrate (Daniel, et al., 2003, Journal of the American Chemical Society, 125(9), pp. 2617-2628). The derivatization does not alter the pharmaceutical activity of enalapril. Previous studies shown that esterification of the prolyl carboxylic acid of enalapril did not affect its activity.

As for lisinopril, the gold nanoparticles modification is performed through exchange of the citrate molecules around the gold nanoparticles by the thiolated enalapril. Because the sulfur-gold interaction is stronger than nitrogen-gold interaction, the stability of the enalapril-capped gold nanoparticles is greater. Since derivatized enalapril does not have negatively charged group, an intermediate is used for the ligand exchange on the gold nanoparticles. Indeed, for modification of citrate-coated gold nanoparticles with neutral or cationic ligands, it was shown that an intermediate exchange of citrate by thioctic acid was necessary to obtain stable modified gold nanoparticles (Lin, et al., 2004, Journal of Physical Chemistry B, 108(7), pp. 2134-2139).

The same characterization techniques described for lisinopril-coated gold nanoparticles was used. Before ligand quantification, the release of the thiolated enalapril from the gold nanoparticles was performed through dissolution of the gold core by potassium cyanide (Liu, et al., 2007, Analytical Chemistry, 79(6), pp. 2221-2229). Since the release of the ligands produces a spontaneous mixed oxidative dimerization of the thiols in air, the calibration curves for LC-MS are done using the corresponding disulfide of thiolated enalapril.

### Example 13

### Biodistribution of Gold Nanoparticle-ACE Inhibitor Conjugates in Transgenic Rats Overexpressing Cardiac ACE

All experiments are performed under the regulations of the Animal Care Committee at the University of Maryland Medical Center, Baltimore, Md., in accordance with the “Guiding Principles in the Care and Use of Animals” as approved by the American Physiological Society. Transgenic rats overexpressing cardiac ACE (originally created by Dr. Martin Paul, Institute of Clinical Pharmacology, Charité University Medical School, Berlin, Germany) are used in this study. The ACE-transgenic rats are normotensive and are compared with nontransgenic Sprague-Dawley rats to be used as controls.

50 rats are used in this study. The injection sites are in the tail veins. Rats are sacrificed at 4 different time points corresponding to time of injection (10, 30, 60, and 120 minutes after injection). The whole animals including tails are measured. These methods are consistent with recommendations from the panel on Euthanasia of the American Veterinary Association. All tissue samples are separately imaged.

To determine whether the nanoparticle labeled conjugates target ACE in vivo, a quantitative comparison of the tissue biodistribution of gold nanoparticle in the presence and absence of lisinopril is performed. Six transgenic ACE overexpressing rats per group are used in this study. Measurements are performed at 10, 30, and 60 minutes after administration. The procedure is repeated for non-transgenic rats. All tissue samples are separately imaged.

### Example 14

### Detection of the Gold Nanoparticle Conjugates with Conventional and K-Edge Tuned Computed Tomography

The attenuation of nanoparticle gold is measured in a conventional animal CT using increasing KV steps between 60 and 140 KV. The measurement is repeated with samples of nanoparticle gold until a signal to noise ratio>2 is achieved for each KV setting. The reference concentration of gold-nanoparticles is used to define the positioning of reference bins in a photon counting computed tomography system (Philips, Hamburg, Germany). The detector in a photon counting CT allows the determination of the k-edge changes in gold-nanoparticles by adjusting the detectors sensitivity for two counts just above and below the gold k-edge. The system is capable to deliver quantitative results after the initial calibration. The method allows separation of the attenuation of gold from other materials in vivo by comparing the attenuation changes below and above the k-edge voxel by voxel. Only voxels that are demonstrating a significant signal change are included into the material specific reconstruction of the acquired data.

All measurements are performed in reference to the above described calibration and tuning process. The attenuation changes below and above the k-edge are relative to the absolute concentration of the gold nanoparticles in the volume of interest. As size of the nanoparticles is known it is possible to calculate the absolute concentration of the material and therefore the concentration of lisinopril-coated gold nanoparticles or other imaging agent of the invention in the tissue volume, therefore indicating the number of bindings with the corresponding targeted structure.

The following measurements are performed: measurement of biodistribution of gold labeled complexes in transgenic and standard rats in correlation to the injected concentration; measurement of biodistribution of gold nanoparticles alone in transgenic and standard rats in correlation to the injected concentration; measurement of absolute concentrations of the gold nanoparticles in reference organs of both transgenic and standard rats; development of reference concentrations for gold nanoparticles with dedicated gold core diameter in transgenic and standard rats; statistical analysis of biodistribution of native gold-nanoparticles and conjugates in reference organs (cardiac, lung, kidney, liver, bones) in transgenic and standard rats; and development of targeted imaging reference concentration and clearance tables for the nano-gold labeled molecules using k-edge imaging with photon counting CT.

While the invention has been described with reference to certain embodiments, those skilled in the art will appreciate that modifications may be made without departing from the scope of the invention. All patents and publications cited in this specification are indicative of the level of those skilled in the art to which the invention pertains. All patents and publications herein are incorporated by reference to the same extent as if each publication was specifically indicated as having been incorporated by reference in its entirety.

What is claimed is:

1. A drug labeled gold nanoparticle, wherein said drug interferes with the activity of the renin-angiotensin system.

2. The drug labeled gold nanoparticle of claim 1, wherein said drug is selected from the group consisting of an angiotensin converting enzyme inhibitor and an angiotensin II receptor antagonist.

3. The drug labeled gold nanoparticle of claim 2, wherein said angiotensin converting enzyme inhibitor is selected from the group consisting of lisinopril, enalapril, captopril, fosinopril, quinapril, ramipril, trandolapril, benazepril, moexipril and perindopril.

4. The drug labeled gold nanoparticle of claim 2, wherein said angiotensin II receptor antagonist is selected from the group consisting of candesartan, eprosartan, irbesartan, losartan, olmesartan, telmisartan, and valsartan.

5. The drug labeled gold nanoparticle of claim 1, wherein said drug is attached to said gold nanoparticle via a capping agent.

6. The drug labeled gold nanoparticle of claim 1, wherein said capping agent contains a phosphine group, an amine group or a thiol group.

7. The drug labeled gold nanoparticle of claim 1, wherein said nanoparticle has a diameter of from about 1 nm to about 100 nm.

8. The drug labeled gold nanoparticle of claim 1, wherein said nanoparticle contains oligo(ethylene glycol), poly(ethylene glycol), or derivatives thereof.

9. The drug labeled gold nanoparticle of claim 1, wherein said nanoparticle contains a fluorescence imaging agent.

10. The drug labeled gold nanoparticle of claim 1, wherein said nanoparticle has a circulation time of from about 2 hours to about 6 hours.

11. A method of imaging an individual, comprising the steps of
administering a plurality of the drug labeled gold nanoparticles of claim 1 to said individual; and
imaging the individual with a diagnostic device.

12. The method of claim 11, further comprising:
monitoring delivery of the drug labeled gold nanoparticles to the individual with the diagnostic device; and diagnosing or monitoring the status of the individual.

13. The method of claim 12, wherein the diagnostic device employs an imaging method selected from the group consisting of MRI, optical imaging, optical coherence tomography, X ray, computed tomography, positron emission tomography, or combinations thereof.

14. The method of claim 11, wherein said individual has or is at risk for heart failure, myocardial ischemia, hypertension, atherosclerosis, diabetic nephropathy or cancer.

15. The method of claim 11, wherein said drug is selected from the group consisting of an angiotensin converting enzyme inhibitor and an angiotensin II receptor antagonist.

16. The method of claim 11, wherein said drug is attached to said gold nanoparticle via a capping agent.

17. The method of claim 16, wherein said capping agent contains a phosphine group, an amine group or a thiol group.

18. The method of claim 11, wherein said nanoparticle has a diameter of from about 1 nm to about 100 nm.

19. A kit, comprising the drug labeled gold nanoparticle of claim 1.

20. The kit of claim 19, wherein said drug labeled gold nanoparticle is contained in a pharmaceutically acceptable formulation that can be administered to a mammal.
